(19) United States
(12) Patent Application Publication
(10) Pub. No.: US 2018/0229525 A1
(43) Pub. Date: Aug. 16, 2018

(54) PRINTING METHOD, RESIN MOLDED PRODUCT, AND MEDICAL DEVICE

(71) Applicant: OLYMPUS CORPORATION, Tokyo (JP)

(72) Inventor: Hisamitsu KUWABARA, Tokyo (JP)

(73) Assignee: OLYMPUS CORPORATION, Tokyo (JP)

(21) Appl. No.: 15/953,687

(22) Filed: Apr. 16, 2018

Related U.S. Application Data
(63) Continuation of application No. PCT/JP2016/082790, filed on Nov. 4, 2016.

(30) Foreign Application Priority Data
Nov. 18, 2015 (JP) 2015-226012

(51) Int. Cl. B41M 5/00; A61L 29/06; C09D 11/101; A61B 17/32; A61B 1/00; A61B 1/005; B41M 7/00; B05D 3/06; B05D 3/10; B05D 7/26; B05D 7/02

(52) U.S. Cl. CPC B41M 5/0047; B05D 7/02; C09D 11/101; A61B 17/320016; A61B 1/00137; A61B 1/0052; A61B 1/00059; A61B 1/0011; B41M 7/0081; B41M 5/0011; B41M 5/0023; B05D 3/067; B05D 3/101; B05D 7/26; A61L 29/06

(57) ABSTRACT

This printing method to print a label on a surface of a resin base includes a dissolving process of dissolving a surface of the resin base using an organic solvent; a printing process of printing a label layer using an UV-curable ink to form the label on the surface of the resin base that is dissolved, and an ink-curing process of irradiating the printed label layer by an ultraviolet light.

This application is a continuation based on a PCT International Application No. PCT/JP2016/082790, filed on Nov. 4, 2016, whose priority is claimed on Japanese Patent Application No. 2015-226012, filed on Nov. 18, 2015. The contents of both the PCT International Application and the Japanese Patent Application are incorporated herein by reference.

### BACKGROUND OF THE INVENTION

### Field of the Invention

The present invention relates to a printing method, a resin molded product, and a medical device.

### Description of Related Art

As disclosed in Japanese Patent No. 4009519, PCT International Publication No. WO2011/132544, and Japanese Unexamined Patent Application, First Publication No. 2015-163701, an endoscope is configured by printing a label that indicates a predetermined logo or a model name on a surface of an operation portion which is formed from a resin material.

Since such label is provided on a medical device, it is necessary that the label has high adhesion characteristic and high chemical resistance characteristic. Accordingly, the label is produced from a thermosetting ink through a screen printing or pad printing process of the thermosetting ink and then heating and curing the thermosetting ink.

The production of the endoscope is a high-mix low-volume production process. During the production process of the endoscope, it is necessary to prepare different printing plates for each model and label, and it is necessary to exchange the printing plates

Furthermore, the printing process on the operation portion of the endoscope means a printing process on a curved surface. Accordingly, for different models, even the printing portion is the same, it is possible that the curvature and the deformation ratio are different. Accordingly, during the printing process of the same label, it is necessary to use a different printing plate for a different model, and it is necessary to exchange the printing plate corresponding to the model.

Accordingly, printing the label by an inkjet printing process without the process of exchanging the printing plate is required.

Recently, regarding the printing process on the resin material, an ultraviolet light (UV) curable ink that is cured by being irradiated with the ultraviolet light is utilized as a superior ink having better quick-drying characteristic and workability compared with the thermosetting ink.

In Japanese Unexamined Patent Application, First Publication No. 2015-163701, a system configured to directly print the UV-curable ink on the resin material and cause the UV-curable ink to be cured by using the UV-curable ink applicable for inkjet printer and a recording device such as an inkjet printer is proposed.

### SUMMARY OF THE INVENTION

According to a first aspect of the present invention, a printing method to print a label on a surface of a resin base includes a dissolving process of dissolving a surface of the resin base using an organic solvent; a printing process of printing a label layer using an UV-curable ink to form the label on the surface of the resin base that has been dissolved, and an ink-curing process of irradiating the printed label layer by an ultraviolet light.

According to a second aspect of the present invention, in the printing method according to the first aspect, the resin base may be one of a group consisting of polysulfone, polyphenylsulfone, and polyetheretherketone (PEEK).

According to a third aspect of the present invention, in the printing method according to the first aspect or the second aspect, a principal ingredient of the organic solvent may be one of a group consisting of coal tar naphtha, isophorone, and toluene.

According to a fourth aspect of the present invention, in the printing method according to any of the first aspect to the third aspect, the UV-curable ink may be an UV-curable acrylic ink.

According to a fifth aspect of the present invention, in the printing method according to any of the first aspect to the fourth aspect, the label layer may be printed by an inkjet method in the printing process.

According to a sixth aspect of the present invention, a resin molded product including a resin base with a surface on which a label is printed includes a label layer which is formed by an UV-curable ink used to form the label; and a mixture layer which is formed by a mixture of the resin base and the UV-curable ink between the resin base and the label layer.

According to a seventh aspect of the present invention, according to the resin molded product according to the sixth aspect, a thickness of the mixture layer may be between 200 nanometers and 2500 nanometers.

According to an eighth aspect of the present invention, a medical device includes the resin molded product according to the sixth aspect or the seventh aspect.

According to a ninth aspect of the present invention, in the medical device according to the eighth aspect, the medical device may be an endoscope.

### BRIEF DESCRIPTION OF DRAWINGS

FIG. 1 is a cross section view showing a process of the printing method according to a first embodiment of the present invention.

FIG. 2 is a cross section view showing a process of the printing method according to the first embodiment of the present invention.

FIG. 3 is a cross section view showing a process of the printing method according to the first embodiment of the present invention.

FIG. 4 is a cross section view showing a process of the printing method according to a first embodiment of the present invention and a cross section view showing a resin molded product according to the first embodiment.

FIG. 5 is a view showing a flow chart of the printing method according to the first embodiment of the present invention.

FIG. 6 is a schematic diagram showing a dissolving process of the printing method according to the first embodiment of the present invention.

FIG. 7 is a schematic diagram showing a printing process and an ink curing process of the printing method according to the first embodiment of the present invention.

FIG. 8 is an oblique view showing an endoscope as a medical device according to the first embodiment of the present invention.

### DETAILED DESCRIPTION OF THE INVENTION

A printing method and a resin molded product according to a first embodiment of the present invention will be described with reference with figures.

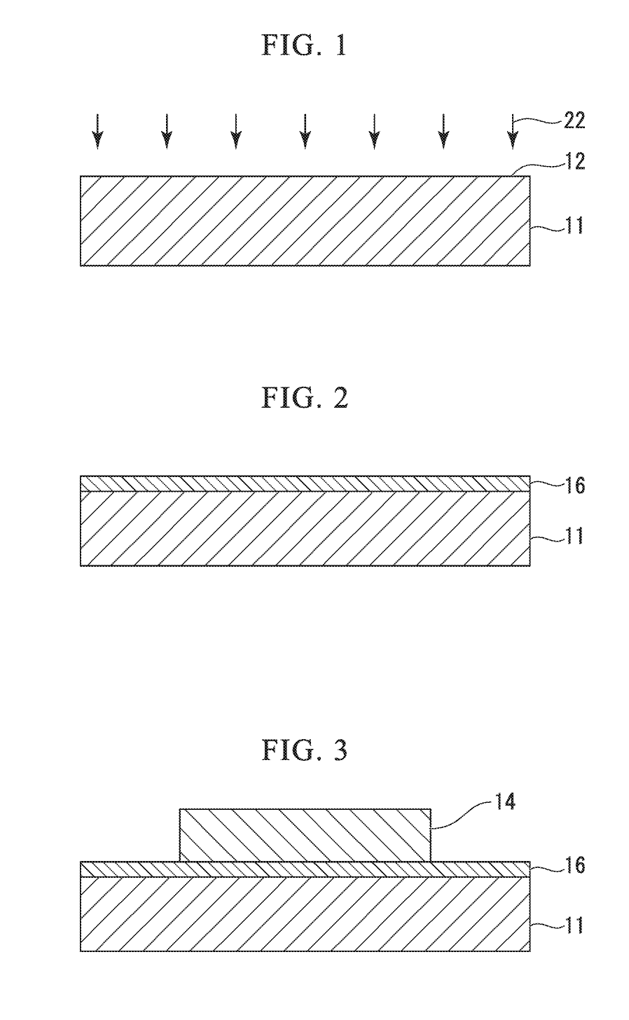

FIGS. 1 to 4 are cross section views showing the printing method according to the present embodiment. In the Figures, the reference sign 10 refers to a resin molded product.

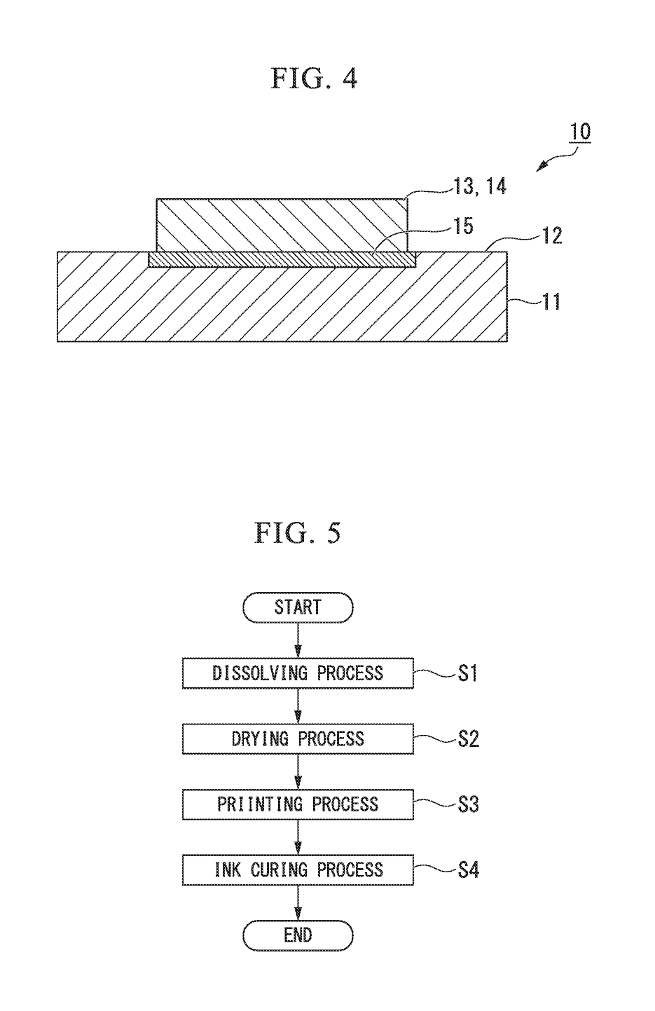

FIG. 4 is a cross section view showing the resin molded product 10 according to the present embodiment.

As shown in FIG. 4, in the resin molded product 10 according to the present embodiment a label 13 is printed on a surface 12 of a resin base 11. The resin molded product 10 has a label layer 14 and a mixture layer 15. The label layer 14 is formed by the UV-curable ink which forms the label 13. The mixture layer 15 is located between the label layer 14 and the resin base 11, and a mixture of the resin of the resin base 11 and the UV-curable ink is existed.

In the present embodiment, the resin base 11 can be formed by one of Polytetrafluoroethylene (PTFE), Tetrafluoroethylene hexafluoropropylene resin (FEP), Polyethylene, polyolefin, polyamide, polyvinyl chloride, latex, natural rubber, polysulfone, polyphenylsulfone, polyetherimide, polyoxymethylene (POM), polyetheretherketone (PEEK), polycarbonate, and acrylonitrile butadiene styrene resin (ABS), and the resin base 11 can be formed by synthetic resins of the resins described above. The resin base 11 is preferably formed by one of the polysulfone, the polyphenylsulfone, and the polyetheretherketone (PEEK).

In the present embodiment, the label layer 14 is formed from the UV-curable ink, and the label layer 14 is a composition which is polymerized and cured once being irradiated by the ultraviolet light (UV) due to the effects of a photopolymerization initiator. The UV-curable ink can be printed by the inkjet method. The UV-curable ink is preferable to be the UV-curable acrylic ink.

The thickness of the label layer 14 is preferably set to be in a range between 5-200 micrometers.

As shown in FIG. 4, the mixture layer 15 is located between the label layer 14 and the resin base 11, and the mixture layer 15 is a composition formed by mixing the resin of the resin base 11 and the UV-curable ink of the label layer 14.

The thickness of the mixture layer 15 is preferably set to be in a range between 200-2500 nanometers.

The existence of the mixture layer 15 can be confirmed by observing the cross sections of the label layer 14 and the resin base 11 using a Transmission Electron Microscope (TEM) while performing a component analysis in the thickness direction thereof.

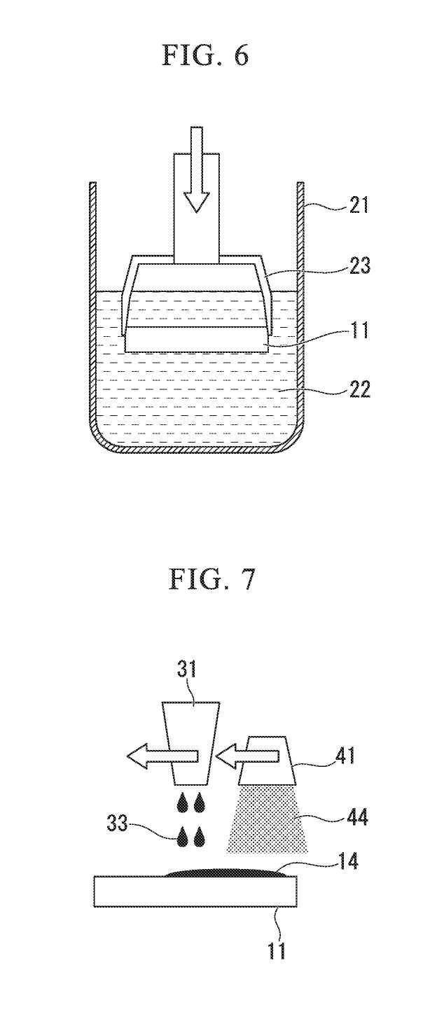

The resin molded product 10 according to the present embodiment is produced based on the printing method according to the present embodiment. FIGS. 1-4 are cross section views showing the process of the printing method according to the present embodiment. FIG. 5 is a flow chart showing the printing method according to the present embodiment. FIG. 6 is a schematic diagram showing a dissolving process of the printing method according to the present embodiment. FIG. 7 is a schematic diagram showing a printing process and an ink curing process of the printing method according to the present embodiment.

The printing method according to the present embodiment, as shown in FIG. 5, includes the dissolving process S1, the drying process S2, the printing process S3, and the ink curing process S4.

As shown in FIG. 1, the dissolving process S1 shown in FIG. 5, is a process of dissolving a surface 12 of the resin base 11 at a predetermined depth by an organic solvent 22, and the dissolving process S1 is a pre-process of the printing process S2. In the dissolving process S1, as shown in FIG. 2, in the surface 12 of the resin base 11 which is partially dissolved by the organic solvent 22, a dissolution layer 16 with a predetermined thickness (depth) is formed. The thickness of the dissolution layer 16 is set so as to make the mixture layer 15 to have a predetermined thickness described below. In order to determine the thickness of the dissolution layer 16, processing conditions such as the type of the organic solvent 22, the exposure time (immersion time) with respect to the organic solvent 22, the processing temperature, and the like are determined.

The organic solvent 22 can be composed to have one of the coal tar naphtha, isophorone, and toluene as a principal ingredient. Here, the “principal ingredient” means that a percentage of the corresponding ingredient is equal to or more than 50% in the solvent. The wording “equal to or more than 50%” may mean a percentage of volume, a percentage of weight, and a percentage of the amount of substance (mole).

The condition of immersing the resin base 11 in the organic solvent 22 can be determined in a period between 1-15 minutes at a temperature between 20-40 degrees Celsius. More preferably, the condition can be determined at a temperature between 35-40 degrees Celsius in a period between 5-10 minutes or 10-15 minutes, and the condition can be determined in accordance with a combination of the organic solvent 22 and the resin base 11.

In the dissolving process S1, any dissolution method which can dissolve the surface 12 of the resin base 11 can be applied and the dissolution method is not limited thereto. For example, the dissolution method can be adopted from a method of immersing the resin base 11 that is held by chucks into the organic solvent 22 stored in a reservoir 21 as shown in FIG. 6, a method of coating the organic solvent on the surface of the rein base 11, or a method of spraying the organic solvent with a predetermined amount on the surface 12 of the rein base 11.

In the surface 12 of the resin base 11, the dissolution layer 16 can be formed only in a region where the label 13 is formed, and the dissolution layer 16 can be formed only in the vicinity of the region. On the other hand, as shown in FIG. 6, in the case in which the resin base is immersed into the organic solvent 22 stored in the reservoir 21, the dissolution layer 16 is formed all over the surface of the resin base 11.

The drying process S2 shown in FIG. 5 is a process of removing the excessive organic solvent 22 from the surface 12 of the resin base 11 until the printing is possible to be performed in the printing process S3 as a post-process. A removing method of removing the organic solvent 22 can be selected and adopted from a method of making the solvent to flow down due to the gravity, a method of vaporizing the organic solvent by setting the temperature and the pressure, a method of warm air drying, a method of cold air drying, a method of wiping the solvent, or a method of scraping, and the like, in a resting state of the resin base 11.

In the drying process S2, as shown in FIG. 2, a state in which the dissolution layer 16 with a predetermined thickness is kept, is determined by the drying conditions such as the temperature, the drying time, the removing method, and the like.

As shown in FIG. 3, the printing process S3 in FIG. 5 is a process of printing the label layer 14 as the label 13 by the UV-curable ink on a surface of the dissolution layer 16 of the resin base 11. The preferable printing method in the printing process S3 is the inkjet printing method. As shown in FIG. 7, the printing method can be adopted as an inkjet printing method of discharging inkjet ink 33 by an inkjet nozzle 31 of an inkjet printer and the like to the surface 12 of the resin base 11.

Accordingly, it is easy to print on the surface of the dissolution layer 16 in a planar state or a curved state. Also, it is possible to form the label 13 corresponding to various surface shapes by printing the label 13 without preparing various printing plates in advance.

The UV-curable ink that is printed in the printing process S3 is mixed with the resin that is dissolved on the surface of the dissolution layer 16 to form the mixture layer 15.

As shown in FIG. 7, the ink curing process S4 in FIG. 5 is a process of curing the UV-curable ink of the label layer 14 and the mixture layer 15 by irradiating a region including the label layer 14 with ultraviolet light 44 having a predetermined wavelength from a UV radiation device 41, immediately after the label 13 is printed in the printing process S3. Accordingly, the label layer 14 and the mixture layer 15 are cured on the surface of the resin base 11.

At the same time, in a region of the dissolution layer 16 where the label layer 14 is not formed, the mixture layer 15 is not formed and the organic solvent is dried such that the surface of the resin base 11 is exposed.

According to the processes described above, as shown in FIG. 4, the resin molded product 10 having the label layer 14 and the mixture layer 15 adhered to the surface 12 of the resin base 11 can be produced.

In the resin molded product 10 according to the present embodiment, the label layer 14 and the resin base 11 are fixedly adhered with each other through the mixture layer 15, and thus the resin molded product 10 can be applied to a medical device required for high adhesion characteristic and high chemical resistance characteristic.

According to the printing method of the present embodiment, it is possible to produce the resin molded product 10 having the label layer 14 described above based on the inkjet method. Accordingly, the workability with regard to the pre-process and the post-process of forming the label layer 14 can be improved and the production cost can be reduced.

Next, a medical device according to the present embodiment will be described.

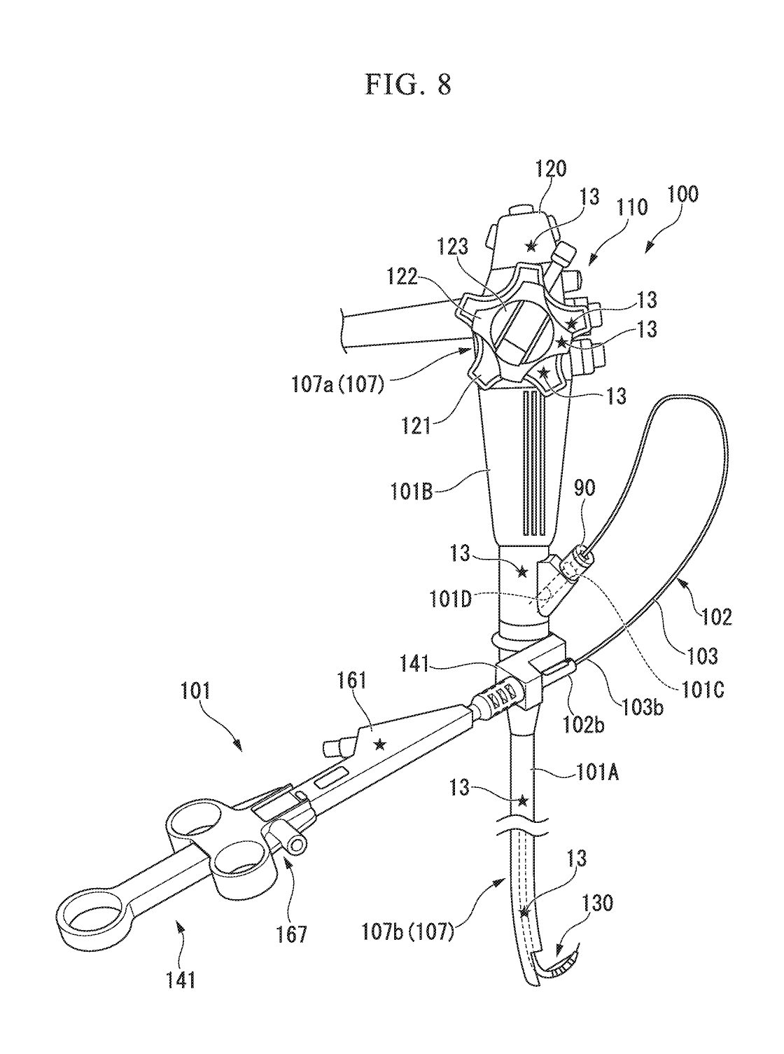

FIG. 8 is an oblique view showing an endoscope (an endoscopic treatment system) as a medical device according to the present embodiment.

The medical device according to the present embodiment is an endoscope (an endoscopic treatment system) 100 having a surface on which the resin molded product is formed.

As shown in FIG. 8, the endoscope (endoscopic treatment system) 100 according to the present embodiment has an endoscope device 110, and an endoscopic treatment tool 101 that is used together with the endoscope device 110 and is configured to perform an excision of the tissues and the like inside the body. The endoscope device 110 and the endoscopic treatment tool 101 are combined with each other to configure a resection system.

As shown in FIG. 8, the endoscope device 110 according to the present embodiment has a tubular member 101A, a grasping portion 101B, a forceps plug 101C, a treatment device channel 101D, and a bending mechanism 107. The tubular member 101A is inserted into the body. The grasping portion 101B is disposed at a proximal side of the tubular member 101A. The forceps plug 101C is disposed at a part of the grasping portion 101B. The treatment device channel 101D is disposed inside the tubular member 101A and the treatment device channel 101D is configured to communicate with the forceps plug 101C. The bending mechanism 107 has a bending operation portion 107a configured to bend a distal part of the tubular member 101A and a bending deformation portion 107b.

The grasping portion 101B is a portion grasped by the operator and an operation portion of the endoscope device 110. The grasping portion 101B has a switch box 120, bending operation knobs 121 and 122, and a rotation-position fixing knob 123. The bending operation knobs 121 and 122 are angle operation members configured to bend a bendable portion disposed at the tubular member 101A. The rotation-position fixing knob 123 is configured to fix a rotation position of the bending operation knob 122.

The endoscopic treatment tool 101 has an insertion potion 102 and an operation portion 140.

The operation portion 140 is a portion grasped by the operator and the operation portion 140 is disposed at a proximal end 102b of the insertion portion 102 (a proximal end 103b of the sheath 103). The operation portion 140 is configured to input various operations in order to operate the endoscopic treatment tool 101.

The operation portion 140 has a distal portion 141, a proximal portion 161, and a handle 167.

The insertion portion 102 is an elongated member that is inserted into the treatment device channel 101D of the endoscope device 110. The insertion portion 102 has the sheath 103 and a knife wire 130.

As shown in FIG. 8, in the endoscopic treatment system (endoscope) 100 according to the present embodiment, the grasping portion 101B, the switch box 120, the bending operation knobs 121 and 122, the rotation-position fixing knob 123, the tubular member 101A, the proximal portion 161 of the operation portion 140, and the like can be configured by the resin molded product 10, wherein the resin molded product 10 is produced by the resin base 11 described above, or the surface of the resin molded product 10 is covered by the resin base 11.

The label 13 configured to be used for the operation of the endoscope 100, or the label 13 configured to indicate the model number of the endoscope 100 is provided on the surface of the resin base 11. The position where the label 13 is provided is not limited to the positions disclosed in FIG. 8.

As shown in FIG. 8, in the endoscopic treatment system (endoscope) 100 according to the present embodiment, the label 13 provided in the grasping portion 101B, the switch box 120, the bending operation knobs 121 and 122, the rotation-position fixing knob 123, the tubular member 101A, the proximal portion 161 of the operation portion 140, and the like can be produced by the printing method described above.

Specifically, each component of the endoscope device 110 such as the grasping portion 101B, the switch box 120, the bending operation knobs 121 and 122, the rotation-position fixing knob 123, the tubular member 101A and the like, or each component of the endoscopic treatment tool 101 such as the proximal portion 161 of the operation portion 140 is composed by the resin molded product 10 having the resin base 11 on the surface thereof.

In the dissolving process S1, the surface 12 of the resin base 11 is dissolved by the organic solvent 22 to form the dissolution layer 16 with a predetermined thickness. In the dissolving process S1, as shown in FIG. 6, it is possible to form the dissolution layer 16 on all over the surface 12 by immersing the resin molded product 10 for forming each component into the organic solvent 22 stored in the reservoir 21, or form the dissolution layer 16 only in a region where the label 13 is formed on the surface 12.

Next, in the drying process S2, the excessive organic solvent 22 is removed from the surface 12 of the resin base 11 until the printing is possible to be performed in the printing process S3 as a post-process. In the drying process S2, as shown in FIG. 6, it is possible to remove the organic solvent and dry the resin base 11 by blowing the warm air to the resin base 11 in a state in which the resin base is held by the chucks.

Next, in the printing method S3, as shown in FIG. 7, the label layer 14 is printed in a region on the surface of the dissolution layer 16 to have a predetermined surface contour shape by discharging the inkjet ink 33 formed from the UV-curable ink by the inkjet nozzle 31 of the inkjet printer and the like according to the inkjet method (see FIG. 3).

In the printing process S3, the printed UV-curable ink is mixed with the resin dissolved on the surface of the dissolution layer 16 to form the mixture layer 15 (see FIG. 4).

Next, in the ink curing process S4, as shown in FIG. 7, the UV-curable ink in the label layer 14 and the mixture layer 15 is cured by irradiating the region including the label layer 14 with the ultraviolet light 44 having the predetermined wavelength. Accordingly, the label layer 44 and the mixture layer 15 are cured on the surface of the resin base 11.

As described above, the resin molded product 10 is produced such that the label 13 formed from the label layer 14 and the mixture layer 15 are adhered on the surface 12 of the resin base 11, and thus each component of the endoscope device 110 such as the grasping portion 101B, the switch box 120, the bending operation knobs 121 and 122, the rotation-position fixing knob 123, the tubular member 101A and the like, or each component of the endoscopic treatment tool 101 such as the proximal portion 161 of the operation portion 140 is produced.

In the endoscopic treatment system (endoscope) 100 according to the present embodiment, the label layer 14 and the resin base 11 are fixedly adhered with each other through the mixture layer. Accordingly, with regard to the endoscopic treatment system (endoscope) 100 according to the present embodiment, the deterioration of the label 13 due to a process of wiping with the alcohol and the like and a scratch caused in the use does not happen such that the endoscopic treatment system 100 can be suitably used as a medical device required for high adhesion characteristic and high chemical resistance characteristic.

Also, the label layer 14 can be formed without exchanging the printing plate that is necessary for the method using the thermosetting ink even in the high-mix low-volume production process of the endoscopic treatment system (endoscope) 100. Accordingly, the workability in the pre-process and the post-process of forming the label layer 14 can be improved, and the production cost can be reduced.

In the present embodiment, each component of the endoscope device 110 such as the grasping portion 101B, the switch box 120, the bending operation knobs 121 and 122, the rotation-position fixing knob 123, the tubular member 101A and the like, or each component of the endoscopic treatment tool 101 such as the proximal portion 161 of the operation portion 140 is selectively described; however, the present invention is not limited thereto. In a case that the label 13 is formed on the surface of other component that is formed from the resin base 11, the present embodiment is also applicable.

The endoscopic treatment tool 101 is described as a configuration for cutting out the tissues, however, the endoscopic treatment tool 101 can be a configuration for other treatment.

### EXAMPLE

Next, an example of the present invention will be described.

The resin base 11 formed from the polysulfone resin is prepared as the resin molded product 10, as the pre-process, the resin base 11 is immersed into the organic solvent 22 and the surface 12 of the resin base 11 is dissolved to form the dissolution layer 16.

For comparison, the surface 12 of the same resin base 11 is wiped and cleaned by ethanol without dissolving ability as the pre-process.

These resin bases 11 are dried in the drying process, and then in the printing process, the label layer 14 and the mixture layer 15 are formed by printing the UV-curable ink based on the inkjet method. Immediately after that, in the ink curing process, the label layer 14 and the mixture layer 15 are irradiated by the ultraviolet light and cured to compose samples having the label layer 14 and the mixture layer 15 fixedly adhered on the surface of the resin base 11.

Coal tar naphtha, isophorone, toluene, and a mixture of these solvent are used as the organic solvent 22. The details of each organic solvent is shown in Tab. 1 as “pre-processing”.

The condition of immersing the resin base 11 into the organic solvent 22 is determined to be at a temperature of 40 degrees Celsius and during a period between 5-15 minutes. The immersing condition can be independently determined according to the type of the organic solvent 22.

The adhesion characteristic and the chemical resistance characteristic of these samples are confirmed. The results are shown in Tab. 1.

Two test method described below are adopted as the evaluation methods.

Test Method 1 [cross-cut test]: The cross-cut test is carried out based on the standard JIS K5600. Tab. 2 is used for a grading evaluation, and a verification standard is determined as the samples from the classification 0 to the classification 2.

Test Method 2 [alcohol wiping test]: The alcohol wiping test is carried out by wiping the printing surface with a gauze containing the ethanol. One reciprocating movement of the gauze is counted as one time. The wiping is repeated by 3000 times and whether the flaking happens or not is confirmed. A verification standard is determined as that none flaking is confirmed after 1000 times of the wiping.

TABLE 1

| | Experimental | Experimental | Experimental | Experimental | Experimental |
|---|---|---|---|---|---|
| | sample 1 | sample 2 | sample 3 | sample 4 | sample 5 |
| pre-processing | wiped with | coal tar | isophorone | toluene | coal tar naphtha: |
| | ethanol only | naphtha | | | isophorone |
| | | | | | 1:1 liquid mixture |
| cross-out | classification | classification | classification | classification | classification |
| test result | 5 | 2 | 0 | 1 | 0 |
| wiped with alcohol | flake at | flake at | none flaking | none flaking | none flaking |
| at 3000 time | 200 times | 2000 times | confirmed | confirmed | confirmed |

According to these results, it can be understood that in the resin base 11 of the present invention, the surface 12 of the resin molded product 10 printed based on the inkjet method and the label layer 14 formed from the UV-curable ink are composed via the mixture layer 15 such that the adhesion characteristic of the resin base 11 and the label layer 14 and the chemical resistance characteristic are improved, and thus the high adhesion characteristic and high chemical resistance characteristic required for a medical device is realized. The present invention is not limited by the above description, and is limited only by the appended claims.

An application example of the present invention can be given not only for an endoscope, but also for a medical device such as a high-frequency medical device or an ultrasonic device for a surgical operation.

The printing method according to the embodiment of the present invention is a printing method of printing a label on a resin base, the printing method has a dissolving process of dissolving a surface of the resin base by an organic solvent, a printing process of printing a label layer using an UV-curable ink to form the label on the dissolved surface of the resin base, and an ink-curing process of irradiating the printed label by an ultraviolet light. According to the printing method described above, the process of printing the UV-curable ink is after the surface of the resin base is dissolved in the dissolving process such that a mixture layer, in which both the UV-curable ink and the resin of the resin base are mixed with each other, exists between the label layer formed from the UV-curable ink and the resin base, and thus the adhesion characteristic of the resin base and the label layer and the chemical resistance characteristic are improved.

Accordingly, it is possible to form the label on the surface of the resin base without using the thermosetting ink which is easy to clog up in the printing device. As a result, the exchange of the printing plate which is necessary in the printing process of the label using the thermosetting ink becomes unnecessary, and thus the work efficiency can be improved. Also, the production process and the production cycle can be shortened to achieve both of the reduction of the production cost and the improvement of the production efficiency.

In the printing method described above, the resin base is one of polysulfone, polyphenylsulfone, and polyetheretherketone (PEEK) such that the surface can be processed to the predetermined dissolved state due to the dissolving process while the favorable mixed state of the resin base and the UV-curable ink can be realized.

In the printing method described above, the organic solvent has one of coal tar naphtha, isophorone, and toluene as a principal ingredient such that the surface of the resin base can be processed to the predetermined dissolved state due to the dissolving process while the favorable mixed state of the resin base and the UV-curable ink can be realized.

The UV-curable ink is the UV-curable acrylic ink such that the formation of the label on the surface of the resin base based on the inkjet printing and the favorable mixed state of the UV-curable ink and the resin base can be realized in a favorable state with regard to the quick-drying characteristic and the workability.

In the printing method described above, the inkjet printing method is adopted in the printing process such that the clogging at the inkjet nozzle when using the thermosetting ink can be prevented. Accordingly, the process of exchanging the printing plate when using the thermosetting ink can be omitted.

The resin molded product according to the embodiment of the present invention is a resin molded product having a resin base with a surface on which a label is printed. The resin molded product has a label layer which is formed by an UV-curable ink used to form the label; and a mixture layer which is formed by a mixture of a resin and the UV-curable ink between the resin base and the label layer. In the resin molded product described above, the mixture layer is formed such that the adhesion characteristic of the label layer and the resin base and the chemical resistance characteristic can be improved. Due to the printing process based on the inkjet method, the label layer can be formed without using the thermosetting ink which is easy to clog in the printing device. Accordingly, the label can be formed based on the inkjet printing method without using the printing plate. As a result, the process of exchanging the printing plates is not necessary such that the label can be formed on the surface of the resin base with a low cost and a favorable workability by reducing the production process.

In the resin molded product described above, the thickness of the mixture layer is in the range of 200-250 nanometers such that the adhesion characteristic of the label layer and the resin base and the chemical resistance can be improved. The thickness of the label layer can be determined in the range of 5-200 micrometers.

In the medical device according to the embodiment of the present invention, the necessary high adhesion characteristic and high chemical resistance characteristic can be achieved by forming any one of the resin molded product described above on the surface thereof.

In the medical device according to the embodiment of the present invention, the medical device can be an endoscope.

The embodiments of the invention have been described above with reference to the drawings, but specific structures of the invention are not limited to the embodiments and may include various modifications without departing from the scope of the invention. The invention is not limited to the above-mentioned embodiments and is limited only by the accompanying claims.

What is claimed is:

1. A printing method to print a label on a surface of a resin base, the printing method comprising:
a dissolving process of dissolving a surface of the resin base using an organic solvent;
a printing process of printing a label layer using an UV-curable ink to form the label on the surface of the resin base that has been dissolved, and
an ink-curing process of irradiating the printed label layer by an ultraviolet light.

2. The printing method according to claim 1, wherein the resin base is one of a group consisting of polysulfone, polyphenylsulfone, and polyetheretherketone (PEEK).

3. The printing method according to claim 1, wherein a principal ingredient of the organic solvent is one of a group consisting of coal tar naphtha, isophorone, and toluene.

4. The printing method according to claim 1, wherein the UV-curable ink is an UV-curable acrylic ink.

5. The printing method according to claim 1, wherein the label layer is printed by an inkjet method in the printing process.

6. A resin molded product including a resin base with a surface on which a label is printed, the resin molded product comprising:
a label layer which is formed by an UV-curable ink used to form the label; and
a mixture layer which is formed by a mixture of the resin base and the UV-curable ink between the resin base and the label layer.

7. The resin molded product according to claim 6, wherein a thickness of the mixture layer is between 200 nanometers and 2500 nanometers.

8. A medical device which includes the resin molded product according to claim 6 formed on a surface of the medical device.

9. The medical device according to claim 8, wherein the medical device is an endoscope.
